(19) United States
(12) Patent Application Publication
(10) Pub. No.: US 2025/0151768 A1
(43) Pub. Date: May 15, 2025

(54) Nutritional Compositions

(71) Applicant: Mardelle Helen Gamble, Calgary (CA)

(72) Inventor: Mardelle Helen Gamble, Calgary (CA)

(21) Appl. No.: 19/023,805

(22) Filed: Jan. 16, 2025

Related U.S. Application Data
(63) Continuation-in-part of application No. 17/615,914, filed on Dec. 2, 2021.

(51) Int. Cl. A23L 33/00; A61K 35/00; A61K 36/185; A61K 36/537; A61K 38/16

(52) U.S. Cl. CPC A23L 33/40; A61K 36/3482; A61K 36/537; A61K 38/168; A23V 2400/175; A61K 2035/115

(57) ABSTRACT

Provided are nutritional compositions having a non-soy-based plant protein component that includes chia proteins. Further disclosed are nutritional compositions having a non-soy-based plant protein source at a level of about 1 g/100 Kcal to about 9.5 g/100 Kcal, wherein at least about 30% of the protein source is a source of chia protein having a protein level of at least about 50% and a fiber level no greater than about 6%. The nutritional compositions disclosed are suitable for administration to human subjects, such as infants.

### RELATED APPLICATION

This application is a continuation-in-part of copending application serial number 17/615,914, filed Dec. 2, 2021 and entitled “Nutritional Compositions” which in turn is a § 371 of PCT/CA2020/050839, the disclosures of each of which are incorporated herein in their entirety.

### TECHNICAL FIELD

The present disclosure relates generally to nutritional compositions, including nutritional beverages, that contain non-soy-based plant proteins, including chia protein, hemp protein, or combinations thereof. The nutritional compositions are infant formulas in some embodiments, and may provide beneficial health effects.

### BACKGROUND ART

One of the main pillars of gastrointestinal health relates to intestinal permeability. Intestinal permeability is the ability of a substance to pass through a specific structure: the human intestines. Villi are finger like projections that line the intestines and hold tightly to one another through tight junctions. Even in a healthy human intestine, certain ingested components can permeate through these tight junctions.

It is a normal function of the intestine to exhibit some permeability, to allow some nutrients to permeate out of the gut, while also maintaining a barrier function to keep potentially harmful substances from leaving the intestine and migrating into the body where they are recognized by the immune system as not belonging to self. The gut-associated lymphoid tissue (GALT) is a critical component of our immune system, protecting our body from foreign invasion. When tight intestinal junctions loosen, and the gut becomes overly permeable, the phenomenon referred to as the “leaky gut” can occur.

Appreciation has been given to the fact that when increased amounts of harmful substances gain access to the body because of a leaky gut barrier, then both local gut and systemic disease elsewhere in the body can result. Such disease can include atopic disease such as eczema, food allergies, celiac disease, type 1 diabetes, asthma, and inflammatory bowel disease.

The cells that line the gastrointestinal tract join through junctional complexes. Three components—the tight junctions (TJ); adherens junctions (AJ); and desmosomes all have occlusive properties, but it is the TJ, sometimes referred to as the zonula occludens, that represents the major barrier between cells. Combination proteins that make up the TJ have been found to function together to act as major gatekeepers. These so called “sealing proteins” act together to keep TJ tight and to decrease intestinal permeability.

Many currently available foods include sucrose monoester fatty acid, a food grade surfactant, that decreases what is called trans epithelial electric resistance (TER) and leads to structural separation of tight junctions in Caco-2 cells. This structural separation can lead to uptake of food particles that the body sees as foreign and therefore mounts an inflammatory response (it is this inflammatory response that is then thought to contribute to disease). In addition, sodium caprate (C10), a medium chain lipid, results in intestinal hyper-permeability and dilation of tight junctions through what is called the phospholipase C dependent inositol triphosphate pathway. These compounds are both found as additives even in hypoallergenic infant formulas. Both sucrose monoester fatty acid and sodium caprate negatively affect the tight junctions which increases intestinal permeability which is being linked to disease.

Some of the proteins in cow's milk and soy are known allergens, and some proteins in cow's milk can be hard to digest for some individuals, including newborns, and is a known risk factor for necrotizing enterocolitis (NEC), a serious condition that develops in the neonatal ICU. NEC is a severe form of intestinal permeability; studies have shown that NEC intestinal tissue has increased intestinal permeability, even at grossly healthy-appearing resection areas. The increase in intestinal permeability in NEC may be related in part to a decrease in occluding proteins. Moreover, the cumulative estrogenic effects of soy have led to concerns that soy may not be a suitable alternative to those children unable to tolerate cow's milk-based infant nutrition. Soy formulas raise concerns of infant and toddlers being exposed to high levels of phytoestrogens. Phytoestrogens are plant-derived substances with estrogenic activity and have been implicated in some studies to have negative effects on the female reproductive system. For example, research has shown observed differences in uterine thickness among soy vs cow milk based formula, onset of early menarche in soy formula use, and more concerning findings of differences in DNA methylation in vaginal cells depending on formula type. Further research has shown that some women fed soy formula as infants were more likely to develop larger uterine fibroids than those not fed soy formula. There is also some evidence that this increased phytoestrogen exposure may have negative effects on an infant's thyroid, particularly in congenital hypothyroidism. Therefore if it is necessary or desirable to feed an infant a vegetable protein based formula, it would be preferable to avoid the potential adverse effects of soy protein phytoestrogens. In addition, soy plants readily accumulate manganese from soil, and soy-based infant formula can contain manganese levels 200 times higher than is found in breast milk. These high levels of manganese have led to possible concerns of adverse effects on child neurodevelopment. However, most commercially available infant formula products and adult meal replacements include dairy or soy.

Cow's milk with its' electrolyte load has the potential to have a renal solute load three times higher than that of human milk. This high renal solute load leads to higher urine osmolar concentration than is observed during feeding with breast milk. When fluid intakes are low and/or when extra-renal water losses are high such as with vomiting and/or diarrhea, the renal concentrating ability of infants may be insufficient for maintaining water balance, and dehydration may be the end result.

Thus, the need for an infant formula and meal replacement/supplement that does not have the disruptors of the tight junctions and further contribute to enhancement of mucosal barrier function through repair of TJ, while also avoiding allergenicity and high electrolyte load, and reducing the cumulative estrogen effects, often observed with cow's milk and/or soy-based compositions.

### BRIEF SUMMARY

Briefly, the present disclosure is directed, in an embodiment, to a nutritional composition that contains a protein component or protein source including non-soy derived plant proteins, comprising chia proteins. In some embodiments, the protein component or protein source also includes amino acids. At least 30% of the protein source is chia protein.

In some embodiments, the nutritional composition includes the protein component described herein in combination with any or all of prebiotics, long chain polyunsaturated fatty acids, one or more probiotics, such as Lactobacillus rhamnosus GG, fats or lipids, and carbohydrates, and can be fully or partially nutritionally complete.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

### DETAILED DESCRIPTION

Reference now will be made in detail to the embodiments of the present disclosure, one or more examples of which are set forth herein below. Each example is provided by way of explanation of the nutritional composition of the present disclosure and is not a limitation. In fact, it will be apparent to those skilled in the art that various modifications and variations can be made to the teachings of the present disclosure without departing from the scope of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are apparent from the following detailed description. It is to be understood by one of ordinary skill in the art that the present discussion is a description of exemplary embodiments only and is not intended as limiting the broader aspects of the present disclosure.

The present disclosure relates generally to nutritional composition having a non-soy-based plant protein component or protein source that includes chia protein. The protein component or protein source can also include hemp protein in certain embodiments. In some embodiments, the protein component or protein source also includes amino acids.

“Nutritional composition” means a substance or formulation that satisfies at least a portion of a subject's nutrient requirements. The terms “nutritional(s)”, “nutritional formula(s)”, “enteral nutritional(s)”, and “nutritional supplement(s)” are used as non-limiting examples of nutritional composition(s) throughout the present disclosure. Moreover, “nutritional composition(s)” may refer to liquids, powders, gels, pastes, solids, tablets, capsules, concentrates, suspensions, or ready-to-use forms of enteral formulas, oral formulas, formulas for infants, formulas for pediatric subjects, formulas for children, growing-up milks and/or formulas for adults.

“Pediatric subject” means a human less than 13 years of age. In some embodiments, a pediatric subject refers to a human subject that is between birth and 8 years old. In other embodiments, a pediatric subject refers to a human subject between 1 and 6 years of age. In still further embodiments, a pediatric subject refers to a human subject between 6 and 12 years of age. The term “pediatric subject” may refer to infants (preterm or fullterm) and/or children, as described below.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes low birth weight infants, very low birth weight infants, and preterm infants. “Preterm” means an infant born before the end of the 37^{th} week of gestation. “Full term” means an infant born after the end of the 37^{th} week of gestation.

“Child” means a subject ranging in age from 12 months to about 13 years. In some embodiments, a child is a subject between the ages of 1 and 12 years old. In other embodiments, the terms “children” or “child” refer to subjects that are between one and about six years old, or between about seven and about 12 years old. In other embodiments, the terms “children” or “child” refer to any range of ages between 12 months and about 13 years.

“Infant formula” means a composition that satisfies the nutrient requirements of an infant. Other countries may have other terms for this composition. For instance, in Canada, the term “human milk substitute” is employed. For the purposes of this disclosure, the term “infant formula” will be meant to include “human milk substitute” or any other term meant to describe a composition that satisfies the nutrient requirements of an infant. In many countries, regulations define the content of an infant formula. “Infant formula” also includes starter infant formula and follow-on formula (sometimes called follow-up formula, or FUF).

The term “medical food” refers to enteral compositions that are formulated or intended for the dietary management of a disease or disorder. Also included within the term “medical food” are “FSMPs”—“foods for special medical purpose”, as defined in the regulations of certain countries. A medical food may be a food for oral ingestion or tube feeding (nasogastric tube), may be labeled for the dietary management of a specific medical disorder, disease or condition for which there are distinctive nutritional requirements, and may be intended to be used under medical supervision.

The term “protein component” as described herein may be used interchangeably with “protein source” and generally refers to the various protein sources that may be used in the nutritional composition. Indeed, use of the term “protein” is not limited to only intact proteins and includes; hydrolyzed protein, peptides, and free amino acids.

As noted, the terms “protein”, “protein source”, and “protein component” also encompasses free amino acids. In some embodiments, the amino acids may comprise, but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine and mixtures thereof. In some embodiments, the amino acids may be branched chain amino acids. In certain other embodiments, small amino acid peptides may be included as the protein component of the nutritional composition. Such small amino acid peptides may be naturally occurring or synthesized.

The term “growing-up milk” refers to a broad category of nutritional compositions intended to be used as a part of a diverse diet in order to support the normal growth and development of a child between the ages of about 1 and about 6 years of age.

“Nutritionally complete” means a composition that may be used as the sole source of nutrition, which would supply essentially all of the required daily amounts of vitamins, minerals, and/or trace elements in combination with proteins, carbohydrates, and lipids. Indeed, “nutritionally complete” describes a nutritional composition that provides adequate amounts of carbohydrates, lipids, essential fatty acids, proteins, essential amino acids, conditionally essential amino acids, vitamins, minerals and energy required to support normal growth and development of a subject.

A nutritional composition that is “nutritionally complete” for a full term infant will, by definition, provide qualitatively and quantitatively adequate amounts of all carbohydrates, lipids, essential fatty acids, proteins, essential amino acids, conditionally essential amino acids, vitamins, minerals, and energy required for growth of the full term infant.

A nutritional composition that is “nutritionally complete” for a child will, by definition, provide qualitatively and quantitatively adequate amounts of all carbohydrates, lipids, essential fatty acids, proteins, essential amino acids, conditionally essential amino acids, vitamins, minerals, and energy required for growth of a child.

“Probiotic” means a microorganism with low or no pathogenicity that exerts a beneficial effect on the health of the host.

“Prebiotic” means a non-digestible food ingredient that beneficially affects the host by selectively stimulating the growth and/or activity of one or a limited number of bacteria in the digestive tract that can improve the health of the host.)

The nutritional composition of the present disclosure may be substantially free of any optional or selected ingredients described herein, provided that the remaining nutritional composition still contains all of the required ingredients or features described herein. Unless otherwise specified, the term “substantially free” means that the selected composition may contain less than a functional amount of the optional ingredient, typically less than 0.1% by weight, and also, including zero percent by weight of such optional or selected ingredient.

All percentages, parts and ratios as used herein are by weight of the total composition, unless otherwise specified.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

As used herein, the term “about” should be construed to refer to both of the numbers specified as the endpoint(s) of any range. Any reference to a range should be considered as providing support for any subset within that range.

Accordingly, provided are nutritional compositions that include non-soy-based plant protein sources comprising chia proteins. The protein sources can also comprise hemp proteins in embodiments. By “non-soy-based plant proteins” is meant that the disclosed nutritional compositions are substantially free of soy proteins.

Chia protein is a nutrient-rich, complete protein, rich in omega 3. In addition, soluble fiber in chia seeds can work as a prebiotic supporting the growth of probiotics in the gut, and chia seeds can contain substantially more calcium than cow's milk. Chia seeds are rich in anti-inflammatory phytochemicals, which are helpful in soothing gastrointestinal distress. Chia proteins can be provided in some embodiments in the form of de-hulled chia seeds, concentrated protein powder, chia protein isolate, or combinations thereof. In some embodiments, the chia proteins are provided in a chia protein concentrate. Hemp protein includes edestin and albumin; in fact, hemp is believed to be among the richest sources of edestin and albumin within the plant kingdom (over other sources such as soy protein). These globulin proteins are responsible for enzymatic functions in the blood plasma and for antibody formation, making them useful for strong immune function. Globulin type proteins are the third most abundant proteins in the human body and are critical for many functions including enzymatic reactions and for both the natural/innate and acquired immunity that an individual develops against invading organisms.

In certain embodiments, incorporating a source of chia protein and hemp protein at a level to provide sufficient protein levels for an infant requires a reduction in the fiber content of chia and hemp seeds and/or flour, etc. For instance, using chia protein can result in a fiber content over 10% in some circumstances. This is considered too high for infant formula, where the normal fiber level is commonly between 2-4%. Higher fiber levels can be associated with some adverse effects and lack regulatory approval. Excessive fiber not only precludes an acceptable nutrient profile, but also creates additional challenges in processing. The higher fiber content significantly increases the viscosity of the formula, complicating processes like spray drying.

As such, reducing or removing the fiber from one of chia or hemp (when present) is not sufficient in many embodiments; rather, the fiber should be removed from both the hemp and chia protein sources. For instance, even if a selected hemp protein source, such as a hemp protein concentrate, has low fiber, the resulting chia-hemp formula still limits the level of chia protein in order to maintain a fiber level below about 6% and in certain embodiments below about 4%. Commercially available chia seeds, chia flours, and chia protein concentrates do not have sufficiently high protein levels and sufficiently low fiber levels to meet these needs.

Thus, some embodiments of the present disclosure provide a way to raise the protein concentration/lower the fiber content of chia or hemp flour or protein concentrate to acceptable levels without using undesirable solvents, such as solvents that are not considered appropriate for food processing. By achieving these levels, the non-nutritional elements in the resulting protein source do not overly dilute the fat, protein, or carbohydrate content, ensuring the composition described herein meets regulatory and consumer standards. More specifically, in embodiments, a protein concentration of at least about 50% is achieved which allows for the development of a formula that maintains a fiber content below about 6%. In other embodiments, the protein concentration is greater than about 55%; in still other embodiments a protein concentration of greater than about 60% is achieved. Consequently, a composition may be achieved with a profile of fats, proteins, and carbohydrates that is acceptable to regulatory standards and desirable to consumers.

In an embodiment, the protein content of a chia or hemp protein source is increased and the fiber content decreased through a series of pH adjustments. More specifically, in some embodiments a protein source such as chia or hemp flour is brought to a pH of about 7.5 to 9.5 using a base such as NaOH or CaOH (after hydration, in embodiments). In certain embodiments, the pH is brought to about 8.0 to 9.0. This step can be repeated more than once.

The resulting liquid phase (the “supernatant”) is then collected and pH adjusted to less than about 5.5 and, in some embodiments, to less than about 5.0 using an acid such as citric acid. In embodiments, the pH is adjusted to between about 4.2 and 4.8. The solids are then collected and brought to a more neutral pH (i.e., between about 6.8 to about 7.3) using a base such as NaOH or CaOH, to produce a protein source having an acceptable protein and fiber level.

In some embodiments, other plant protein sources that can be incorporated into the nutritional composition of this disclosure as a protein source, in addition to chia protein, hemp protein, or a combination of the two, include those derived from maize, amaranth, quinoa, oats, flaxseed, chickpeas, fava beans, rice, peas, and lentils. In certain embodiments, the protein component of the disclosed nutritional composition comprises both chia and hemp protein sources.

In embodiments, the protein source includes a source of intact protein. In one embodiment, the protein source of the nutritional composition may include intact proteins, or a combination of both intact proteins and partially hydrolyzed proteins, with a degree of hydrolysis of between about 4% and 20%. In certain other embodiments, the protein source may include a protein that is more completely hydrolyzed. In still other embodiments, the protein source is supplemented with amino acids. In yet another embodiment, the protein source may be supplemented with glutamine-containing peptides.

In some embodiments, the nutritional composition comprises no greater than about 9.5 g/100 Kcal, and, in certain embodiments, between about 1 g and about 9.5 g of total protein sources per 100 Kcal. In other embodiments, the nutritional composition comprises between about 2.0 g and about 8.5 g of protein source per 100 Kcal. Indeed, the amount of total protein sources in the disclosed nutritional composition can vary depending on the end use. For instance, in some embodiments where the nutritional composition is infant formula, the nutritional composition can comprise between about 2.0 g and about 4.8 g of protein source per 100 Kcal; in some embodiments where the nutritional composition is intended for a child, such as a growing up milk, the nutritional composition can comprise between about 6.0 g and about 6.8 g of protein source per 100 Kcal; and in certain embodiments where the nutritional composition is a medical food, the nutritional composition can comprise between about 7.4 g and about 8.3 g of protein source per 100 Kcal

At least about 15% of the protein source is, in some embodiments, a source of chia protein. In other embodiments, a source of chia protein is at least about 30% of the protein source; in yet other embodiments, the protein source is about 50% chia protein source. In certain embodiments, a source of chia protein makes up 100% of the protein source.

Moreover, in some embodiments, a hemp protein source is at least about 20% of the protein source; in still other embodiments, a hemp protein source is at least about 35% of the protein source. In certain embodiments, the nutritional composition includes the combination of chia protein source and hemp protein source at a ratio of chia protein source to hemp protein source which can vary between about 1:4 and about 4:1.

In some embodiments, the nutritional composition also comprises free amino acids; the amino acids may comprise, but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine and mixtures thereof. In certain embodiments, the amino acids included in the nutritional composition of the present disclosure comprise isoleucine, leucine, lysine, threonine, tryptophan, or combinations thereof. The total amount of amino acids included in the nutritional composition can be, in some embodiments, between about 0.1 g per 100 Kcal to about 3.2 g per 100 Kcal. In other embodiments, the total amount of amino acids included in the nutritional composition can be between about 1.1 g per 100 Kcal to about 2.8 g per 100 Kcal. The individual amino acids included can each be included in varying amounts to assist in achieving the desired nutritional profile.

Depending on the protein source, it may be desirable to include amino acids to ensure the full complement of desirable amino acids is present in the nutritional composition of the present disclosure, depending on the age of the subject. In other words, if it is found that the proteins employed, and the levels at which they are employed, do not provide the desired complement of amino acids, such as those found in human breast milk, the protein source can be supplemented with those “missing” amino acids, in an embodiment. Thus, in certain embodiments, amino acid supplementation can be used to ensure amino acid levels are desirable, such as at the levels found in human breast milk, or as otherwise desired based on the purpose for the nutritional composition.

In some embodiments, the nutritional composition of the present disclosure also includes at least one probiotic. In some embodiments, the probiotic comprises Lactobacillus rhamnosus GG (“LGG”) (ATCC 53103). In certain other embodiments, the probiotic may be selected from any other Lactobacillus species, or from Bifidobacterium species, other probiotic species, or any combination thereof.

The amount of the probiotic may vary from about 1×10⁴ to about 1.5×10¹² cfu of probiotic(s) per 100 Kcal of nutritional composition. In some embodiments the amount of probiotic may be from about 1×10⁶ to about 1×10⁹ cfu of probiotic(s) per 100 Kcal. In certain other embodiments the amount of probiotic may vary from about 1×10⁷ cfu/100 Kcal to about 1×10⁸ cfu of probiotic(s) per 100 Kcal.

In some embodiments, the nutritional composition of the present disclosure also comprises prebiotics, which can exert health benefits, which may include selective stimulation of the growth and/or activity of one or more beneficial gut bacteria, stimulation of the growth and/or activity of ingested probiotic microorganisms, selective reduction in gut pathogens, and favorable influence on gut short chain fatty acid profile. The chia protein source in accordance with this disclosure may contain prebiotics (referred to as endogenous prebiotics, because they are present as an element of another component, in this case, chia proteins). Other plant source of protein that may be present in the protein component of the disclosed nutritional composition and which can also function as sources of endogenous prebiotics include hemp, oat, flax, lentils, peas, and chickpeas. In some embodiments, however, the nutritional composition may include additional prebiotics (referred to as exogenous prebiotics). Prebiotics useful in the present disclosure may include oligosaccharides including, but not limited to, human milk oligosaccharides (HMOs) such as 2′-fucosyllactose (2-FL) and sialyl-lactose, polysaccharides, and other prebiotics that contain fructose, xylose, soya, galactose, glucose and mannose.

More specifically, prebiotics useful in the present disclosure include polydextrose, galacto-oligosaccharide, lactulose, fructo-oligosaccharide, inulin, HMOs, and other prebiotics known in the art.

In an embodiment, the total amount of exogenous prebiotics present in the nutritional composition may be from about 1.0 g/L to about 10.0 g/L of the composition. In other embodiments, the total amount of exogenous prebiotics present in the nutritional composition may be from about 2.0 g/L and about 8.0 g/L of the composition. In some embodiments, the total amount of exogenous prebiotics present in the nutritional composition may be from about 0.1 g/100 Kcal to about 1.5 g/100 Kcal. In certain embodiments, the total amount of exogenous prebiotics present in the nutritional composition may be from about 0.15 g/100 Kcal to about 1.5 g/100 Kcal.

The nutritional composition(s) of the present disclosure may also comprise a carbohydrate source. Carbohydrate sources can be dextrose, tapioca syrup, lactose (regular and/or in hypoallergenic form), glucose, fructose, coconut sugar, coconut palm sugar, cane sugar, maple syrup, rice syrup, molasses, maltodextrins, sucrose, starch (such as tapioca starch, rice starch, or corn starch), palm nectar, coconut sap sugar, agave syrup, or combinations thereof, as well as other carbohydrates known in the art, like corn syrup solids. The amount of carbohydrate in the nutritional composition typically can vary from between about 6 g/100 Kcal to about 17 g/100 Kcal. In some embodiments, the amount of carbohydrate is between about 8 g and about 16 g/100 Kcal. In other embodiments, the amount of carbohydrate is between about 9 g and about 16 g/100 Kcal. Moreover, hydrolyzed, partially hydrolyzed, and/or extensively hydrolyzed carbohydrates may be desirable for inclusion in the nutritional composition due to their easy digestibility.

In some embodiments, the nutritional composition described herein comprises a fat or lipid source. In certain embodiments, appropriate fat sources include, but are not limited to, marine sources, such as fish oils, marine oils, single cell oils; vegetable and plant oils, such as sunflower oil, palm kernel oil, coconut oil, high oleic sunflower oil, olive oil, flaxseed (linseed) oil, safflower oil, chia oil, hemp oil, rapeseed oil, palm oil,; medium chain triglycerides; and any combinations thereof, as well as others known in the art.

In some embodiments the nutritional composition comprises between about 2 g/100 Kcal to about 8 g/100 Kcal of a fat or lipid. In some embodiments, the nutritional composition comprises between about 2.4 g/100 Kcal to about 7 g/100 Kcal of a fat source. In other embodiments the fat source may be present in an amount from about 3 g/100 Kcal to about 6.1 g/100 Kcal.

In some embodiments the nutritional composition may also include a source of long chain polyunsaturated fatty acids (LCPUFA). In one embodiment the amount of LCPUFA in the nutritional composition is advantageously at least about 5 mg/100 Kcal, and may vary from about 5 mg/100 Kcal to about 100 mg/100 Kcal, more preferably from about 10 mg/100 Kcal to about 50 mg/100 Kcal. Non-limiting examples of LCPUFAs include, but are not limited to, docosahexaenoic acid (DHA), eicosapentaenoic acid (EPA), arachidonic acid (ARA), linoleic acid (18:2 n-6), alpha-linolenic acid (18:3 n-6), and others known in the art, as well as combinations thereof.

In some embodiments, the LCPUFA included in the nutritional composition may comprise DHA. In one embodiment the amount of DHA in the nutritional composition is advantageously at least about 17 mg/100 Kcal, and may vary from about 5 mg/100 Kcal to about 75 mg/100 Kcal, more preferably from about 10 mg/100 Kcal to about 50 mg/100 Kcal.

In another embodiment, especially if the nutritional composition is an infant formula, the nutritional composition is supplemented with both DHA and ARA. In this embodiment, the weight ratio of ARA:DHA may be between about 1:3 and about 9:1. In a particular embodiment, the ratio of ARA:DHA is from about 1:2 to about 4:1.

If other LCPUFAs are included in the nutritional composition, the levels can be different. For instance, in some embodiments, alpha-linoleic acid can be incorporated at a level of about 40 mg/100 Kcal to about 170 mg/100 Kcal; likewise, in some embodiments, linolenic acid can be included at a level of about 250 mg/100 Kcal to about 1500 mg/100 Kcal.

One or more vitamins and/or minerals may also be added to the nutritional composition in amounts sufficient to supply the daily nutritional requirements of a subject. It is to be understood by one of ordinary skill in the art that vitamin and mineral requirements will vary, for example, based on the age of the subject. For instance, an infant may have different vitamin and mineral requirements than a child between the ages of one and thirteen years, and an adult different vitamin and mineral requirements from either infant or child. Thus, the embodiments are not intended to limit the nutritional composition to a particular age group but, rather, to provide a range of acceptable vitamin and mineral components.

In embodiments providing a nutritional composition for a child, the composition may optionally include, but is not limited to, one or more of the following vitamins or derivations thereof: vitamin B₁ (thiamin, thiamin pyrophosphate, TPP, thiamin triphosphate, TTP, thiamin hydrochloride, thiamin mononitrate), vitamin B₂ (riboflavin, flavin mononucleotide, FMN, flavin adenine dinucleotide, FAD, lactoflavin, ovoflavin), vitamin B₃ (niacin, nicotinic acid, nicotinamide, niacinamide, nicotinamide adenine dinucleotide, NAD, nicotinic acid mononucleotide, NicMN, pyridine-3-carboxylic acid), vitamin B₃-precursor tryptophan, vitamin B₆ (pyridoxine, pyridoxal, pyridoxamine, pyridoxine hydrochloride), pantothenic acid (pantothenate, panthenol), folate (folic acid, folacin, pteroylglutamic acid), vitamin B₁₂ (cobalamin, methylcobalamin, deoxyadenosylcobalamin, cyanocobalamin, hydroxycobalamin, adenosylcobalamin), biotin, vitamin C (ascorbic acid), vitamin A (retinol, retinyl acetate, retinyl palmitate, retinyl esters with other long-chain fatty acids, retinal, retinoic acid, retinol esters), vitamin D (calciferol, cholecalciferol, vitamin D₃, 1,25,-dihydroxyvitamin D), vitamin E (α-tocopherol, α-tocopherol acetate, α-tocopherol succinate, α-tocopherol nicotinate, α-tocopherol), vitamin K (vitamin K₁, phylloquinone, naphthoquinone, vitamin K₂, menaquinone-7, vitamin K₃, menaquinone-4, menadione, menaquinone-8, menaquinone-8H, menaquinone-9, menaquinone-9H, menaquinone-10, menaquinone-11, menaquinone-12, menaquinone-13), choline, inositol, β-carotene and any combinations thereof.

In embodiments providing a children's nutritional product, such as a growing-up milk, the composition may optionally include, but is not limited to, one or more of the following minerals or derivations thereof: boron, calcium, calcium acetate, calcium gluconate, calcium chloride, calcium lactate, calcium phosphate, calcium sulfate, chloride, chromium, chromium chloride, chromium picolonate, copper, copper sulfate, copper gluconate, cupric sulfate, fluoride, iron, carbonyl iron, ferric iron, ferrous fumarate, ferric orthophosphate, iron trituration, polysaccharide iron, iodide, iodine, magnesium, magnesium carbonate, magnesium hydroxide, magnesium oxide, magnesium stearate, magnesium sulfate, manganese, molybdenum, phosphorus, potassium, potassium phosphate, potassium iodide, potassium chloride, potassium acetate, selenium, sulfur, sodium, docusate sodium, sodium chloride, sodium selenate, sodium molybdate, zinc, zinc oxide, zinc sulfate and mixtures thereof. Non-limiting exemplary derivatives of mineral compounds include salts, alkaline salts, esters and chelates of any mineral compound.

The minerals can be added to growing-up milks or to other children's nutritional compositions in the form of salts such as calcium phosphate, calcium glycerol phosphate, sodium citrate, potassium chloride, potassium phosphate, magnesium phosphate, ferrous sulfate, zinc sulfate, cupric sulfate, manganese sulfate, and sodium selenite. Additional vitamins and minerals can be added as known within the art.

The nutritional compositions of the present disclosure may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The nutritional compositions of the present disclosure may optionally include one or more emulsifiers that may be added for stability of the final product. Examples of suitable emulsifiers include, but are not limited to, lecithin (e.g., from egg, soy, or sunflower), alpha lactalbumin and/or mono-and di-glycerides, and mixtures thereof. Other emulsifiers are readily apparent to the skilled artisan and selection of suitable emulsifier(s) will depend, in part, upon the formulation and final product.

In some embodiments, the nutritional composition may be formulated to include from about 0.1 wt % to about 1 wt % of emulsifier based on the total dry weight. of the nutritional composition. In other embodiments, the nutritional composition may be formulated to include from about 0.7 wt % to about 2 wt % of emulsifier based on the total dry weight of the nutritional composition.

The nutritional compositions of the present disclosure may optionally include one or more preservatives that may also be added to extend product shelf life. Suitable preservatives include, but are not limited to, potassium sorbate, sodium sorbate, potassium benzoate, sodium benzoate, potassium citrate, calcium disodium EDTA, and mixtures thereof.

In some embodiments the nutritional composition may be formulated to include from about 0.1 wt % to about 1.5 wt % of a preservative based on the total dry weight of the composition. In other embodiments, the nutritional composition may be formulated to include from about 0.2 wt % to about 1.1 wt % of a preservative based on the total dry weight of the composition.

In some embodiments where the nutritional composition is a ready-to-use liquid composition, the nutritional composition may be formulated to include from about 0.5 g/L to about 5 g/L of preservative. Still, in certain embodiments, the nutritional composition may include from about 1 g/L to about 3 g/L of preservative.

The nutritional compositions of the present disclosure may optionally include one or more stabilizers. Suitable stabilizers for use in practicing the nutritional composition of the present disclosure include, but are not limited to, gum arabic, gum ghatti, gum karaya, gum tragacanth, agar, furcellaran, guar gum, gellan gum, locust bean gum, manioc starch, pectin, low methoxyl pectin, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), polysorbitols, dextran, carrageenans and mixtures thereof.

In some embodiments where the nutritional composition is a ready-to-use liquid composition, the nutritional composition may be formulated to include from about 50 mg/L to about 150 mg/L of stabilizer. Still, in certain embodiments, the nutritional composition may include from about 80 mg/L to about 120 mg/L of stabilizer.

The nutritional compositions of the disclosure may provide minimal, partial or total nutritional support. The compositions may be nutritional supplements or meal replacements. The compositions may, but need not, be nutritionally complete. In an embodiment, the nutritional composition of the disclosure is nutritionally complete and contains suitable types and amounts of lipid, carbohydrate, protein, vitamins and minerals.

In an embodiment, the children's nutritional composition may contain between about 10 and about 100% of the dietary recommendation for any given country. In other embodiments, the children's nutritional composition may contain between about 20 and about 50% of the dietary recommendation for any given country. Of course, the foregoing depends on the expected number of feedings per day. In yet another embodiment, the levels of vitamin D, calcium, magnesium, phosphorus, and potassium in the children's nutritional product may correspond with the average levels found in milk.

In some embodiments the nutritional composition is an infant formula. Infant formulas are fortified nutritional compositions for an infant. The content of an infant formula is often dictated by regulations, which define macronutrient, vitamin, mineral, and other ingredient levels in an effort to simulate the nutritional and other properties of human breast milk. Infant formulas are designed to support overall health and development in a pediatric human subject, such as an infant or a child.

In some embodiments, the nutritional composition of the present disclosure is a growing-up milk. Growing-up milks are fortified milk-based beverages intended for children over 1 year of age (typically from 1-3 years of age, from 4-6 years of age or from 1-6 years of age). They are not medical foods and are not intended as a meal replacement or a supplement to address a particular nutritional deficiency. Instead, growing-up milks are designed with the intent to serve as a complement to a diverse diet to provide additional insurance that a child achieves continual, daily intake of all essential vitamins and minerals, macronutrients plus additional functional dietary components, such as non-essential nutrients that have purported health-promoting properties.

The exact composition of a growing-up milk or other nutritional composition according to the present disclosure can vary from market-to-market, depending on local regulations and dietary intake information of the population of interest. Moreover, nutrient values can vary between markets depending on the identified nutritional needs of the intended population, raw material contributions and regional regulations.

The disclosed nutritional composition(s) may be provided in any form known in the art, such as a powder, a gel, a suspension, a paste, a solid, a liquid, a liquid concentrate, a reconstituteable powdered milk substitute or a ready-to-use product. The nutritional composition may, in certain embodiments, comprise a nutritional supplement, children's nutritional product, infant formula, preterm infant formula, human milk fortifier, growing-up milk or any other nutritional composition designed for a human subject. Nutritional compositions of the present disclosure include, for example, orally-ingestible, health-promoting substances including, for example, foods, beverages, tablets, capsules and powders. Moreover, the nutritional composition of the present disclosure may be standardized to a specific caloric content, it may be provided as a ready-to-use product, or it may be provided in a concentrated form. In some embodiments, the nutritional composition is in powder form.

The powdered nutritional compositions may be formulated with sufficient kinds and amounts of nutrients so as to provide a sole, primary, or supplemental source of nutrition, or to provide a specialized powdered nutritional formulation for use in individuals afflicted with specific diseases or conditions. For example, in some embodiments, the nutritional compositions disclosed herein may be suitable for administration to pediatric subjects and infants in order provide exemplary health benefits disclosed herein. In other embodiments, the nutritional composition may also be suitable for administration to adult humans or even have veterinary uses.

The powdered nutritional compositions provided herein may further comprise other optional ingredients that may modify the physical, chemical, hedonic or processing characteristics of the products or serve as nutritional components when used in the targeted population. Many such optional ingredients are known or otherwise suitable for use in other nutritional products and may also be used in the powdered nutritional compositions described herein, provided that such optional ingredients are safe and effective for oral administration and are compatible with the essential and other ingredients in the selected product form. Non-limiting examples of such optional ingredients include preservatives, antioxidants, emulsifying agents, buffers, additional nutrients as described herein, colorants, flavors, thickening agents and stabilizers, and so forth.

### Protein Isolation Example

137 kgs of chia flour is mixed with 2800 liters of cold tap water in a tank with high agitation. The flour is added manually on top of tank under agitation. Once all the flour is dissolved, the slurry is allowed to hydrate for 30-40 minutes. The pH is then adjusted using 15% NaOH to a pH of 8.3-8.5 following an additional 20 minutes of mixing. The pH adjusted slurry is then fed into a horizontal decanter centrifuge to separate the solids fraction from the liquid fraction. Solids are collected into a tank along with 50° C. water (total amount of water for process: 1400 liters) under slight agitation. The resuspended solids slurry is again pH adjusted to 8.3-8.5 under agitation using 15% NaOH and mixed for 40 minutes. The pH adjusted slurry is then fed to the a horizontal decanter centrifuge. The solids phase is collected in a barrel with minimal dilution water, then weighed and disposed.

The liquid phase is collected and pH adjusted to 4.4-4.5 under gentle agitation using 20% citric acid. The pH adjusted slurry is then kept under slow agitation for 60 minutes to allow the protein curd to precipitate. The slurry is then fed to a horizontal decanter centrifuge. The solids protein curd fraction is collected with minimal water in clean 5 gallon buckets and the liquids are disposed.

Once all solids are collected from the third decanter run. A stainless steel barrel with mixer is used to agitate the solids and take a dry matter sample. 15% NaOH is then used to neutralize the pH of the protein to a target pH of 7.0-7.1 and then fed through a high-temperature/short time direct steam injection pasteurization system following a flash cooling under vacuum. The pasteurized protein slurry is then fed into a tall form high pressure spray dryer to recover 10.2 kg total of chia protein powder. The powder is analyzed and the results are shown in Table 1:

TABLE 1

| Test Parameters | Results | Units |
|---|---|---|
| Moisture | 3.86 | % |
| Protein as N × 6.25 | 67.8 | % |
| Fat | 21.11 | % |
| Ash | 4.07 | % |
| Aerobic Plate Count | 550 | cfu/g |

### Formulation Examples

Formulation examples are provided to illustrate some embodiments of the nutritional composition of the present disclosure but should not be interpreted as any limitation thereon. Other embodiments within the scope of the claims herein will be apparent to one skilled in the art from the consideration of the specification or practice of the nutritional composition or methods disclosed herein. It is intended that the specification, together with the example, be considered to be exemplary only, with the scope and spirit of the disclosure being indicated by the claims which follow the example.

TABLE 1
First Exemplary Formulation

per 100 kcal

| Nutrient | Minimum | Maximum |
|---|---|---|
| Chia protein source (g) | 3 | 4 |
| Free amino acids (g) | 0.7 | 1.4 |
| Carbohydrates (g) | 5 | 13 |
| Fat (g) | 4 | 8 |
| LCPUFA (mg) | 5 | 150 |
| Prebiotics (g) | 0.01 | 1.5 |
| Vitamins, minerals, etc. (mg) | 200 | 600 |

TABLE 2
Second Exemplary Formulation

per 100 kcal

| Nutrient | Minimum | Maximum |
|---|---|---|
| Chia protein source (g) | 0.8 | 1.6 |
| Hemp protein source (g) | 1.7 | 2.5 |
| Free amino acids (g) | 0.2 | 0.6 |
| Carbohydrates (g) | 5 | 13 |
| Fat (g) | 1 | 8 |
| LCPUFA (mg) | 5 | 150 |
| Prebiotics (g) | 0.01 | 1.5 |
| Vitamins, minerals, etc. (μg) | 200 | 600 |

TABLE 3
Third Exemplary Formulation

per 100 kcal

| Nutrient | Minimum | Maximum |
|---|---|---|
| Chia protein source (g) | 3.2 | 4.2 |
| Free amino acids source (g) | 2.4 | 3.2 |
| Carbohydrates (g) | 5 | 15 |
| Fat (g) | 1 | 6 |
| LCPUFA (mg) | 5 | 150 |
| Prebiotics (g) | 0.01 | 1.5 |
| Vitamins, minerals, etc. (μg) | 500 | 900 |

TABLE 4
Fourth Exemplary Formulation

per 100 kcal

| Nutrient | Minimum | Maximum |
|---|---|---|
| Chia protein source (g) | 1.8 | 3.0 |
| Hemp protein source (g) | 2.3 | 3.0 |
| Free amino acids (g) | 1.3 | 2.2 |
| Carbohydrates (g) | 5 | 15 |
| Fat (g) | 1 | 6 |
| LCPUFA (mg) | 5 | 150 |
| Prebiotics (g) | 0.01 | 1.5 |
| Vitamins, minerals, etc. (μg) | 600 | 900 |

TABLE 5
Fifth Exemplary Formulation

per 100 kcal

| Nutrient | Minimum | Maximum |
|---|---|---|
| Chia protein source (g) | 0.9 | 1.8 |
| Hemp protein source (g) | 3.8 | 4.9 |
| Free amino acids (g) | 1.9 | 2.8 |
| Carbohydrates (g) | 10 | 18 |
| Fat (g) | 1 | 5 |
| LCPUFA (mg) | 5 | 150 |
| Prebiotics (g) | 0.01 | 1.5 |
| Vitamins, minerals, etc. (μg) | 600 | 1100 |

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. Therefore, the spirit and scope of the appended claims should not be limited to the description of the versions contained therein.

What is claimed is:

1. A nutritional composition comprising:
a carbohydrate source;
a fat or lipid source; and
a non-soy-based plant protein source at a level of about 1 g/100 Kcal to about 9.5 g/100 Kcal, wherein at least about 30% of the protein source comprises a source of chia protein having a protein level of at least about 50% and a fiber level no greater than about 6%.

2. The composition of claim 1, wherein at least 20% of the protein source comprises hemp protein having a protein level of at least about 50% and a fiber level no greater than about 6%.

3. The composition of claim 1, wherein the protein source is present in an amount of from about 2.0 g/100 Kcal to about 4.8 g/100 Kcal.

4. The composition of claim 1, wherein the protein source further comprises free amino acids.

5. The composition of claim 4, wherein the free amino acids comprise isoleucine, leucine, lysine, threonine, tryptophan, or combinations thereof.

6. The composition of claim 5, wherein free amino acids are included at a level of between about 0.1 g per 100 Kcal to about 3.2 g per Kcal.

7. The composition of claim 1, wherein the nutritional composition further comprises a probiotic.

8. The composition of claim 1, wherein the nutritional composition further comprises a prebiotic.

9. The composition of claim 1, wherein the nutritional composition further comprises one or more long chain polyunsaturated fatty acids.

10. The composition of claim 9, wherein the one or more long chain polyunsaturated fatty acids comprises docosahexaenoic acid, arachidonic acid, eicosapentaenoic acid, linoleic acid, γ-linolenic acid, or combinations thereof.

11. The composition of claim 1, wherein the composition comprises an infant formula.

12. The composition of claim 11, wherein the composition is a powdered infant formula.

13. The composition of claim 11, wherein the composition is a liquid infant formula.
